(19) United States
(12) Patent Application Publication
(10) Pub. No.: US 2001/0049505 A1
(43) Pub. Date: Dec. 6, 2001

(54) Thermistor holder

(76) Inventor: Timothy N. Byrd, Townsend, TN (US)

Correspondence Address: Jeffrey N. Cutler, Pitts & Brittian, P.C., P.O. Box 51295, Knoxville, TN, 37950-1295, US

(21) Appl. No.: 09/906,337

(22) Filed: Jul. 16, 2001

Related U.S. Application Data
(63) Continuation-in-part of application No. 09/417,598, filed on Oct. 14, 1999.
Application No. 09/417,598 is a continuation-in-part of application No. 08/873,495, filed on Jun. 12, 1997, now abandoned.
Application No. 08/873,495 is a continuation-in-part of application No. 08/636,976, filed on Apr. 24, 1996, now Pat. No. 5,676,137.

(51) Int. Cl. A61M 5/32; A62B 9/06; A61M 16/00

(52) U.S. Cl. 604/180; 128/207.14; 604/179

(57) ABSTRACT

A thermistor holder for a holder for a thermistor especially for use with infants. The thermistor holder includes a foundation strap having first and second end portions and upper and lower surfaces. An adhesive surface portion provided on the lower surface of the foundation strap for releasably securing said foundation strap to a supporting object is provided. A bridging member is carried by the foundation strap second end portion and carries an engaging strap for being releasably secured to the patient. The thermistor holder also includes a securing strap, secured at its proximal end portion to the foundation strap and releasably securable at its distal end to a portion of the engaging strap. The securing strap releasably engages at least a portion of the engaging strap, after the thermistor has been received between the securing strap and the bridging member. An insulative member is provided between the securing strap and the bridging portion to insulate the thermistor from ambient temperatures. The upper surface of the insulative member is provided with a reflective surface to further assist in the accuracy of the temperature readings acquired by the thermistor

### CROSS-REFERENCE TO RELATED APPLICATIONS

This application is a Continuation-In-Part of Ser. No. 09/417,598, filed on Oct. 14, 1999, which is a continuation-in-part of my earlier filed application Ser. No. 08/873,495, filed Jun. 12, 1997, which is a continuation-in-part of my earlier filed application Ser. No. 08/636,976, filed Apr. 24, 1996, and which issued on Oct. 14, 1997 as U.S. Pat. No. 5,676,137.

### STATEMENT REGARDING FEDERALLY SPONSORED RESEARCH OR DEVELOPMENT

Not Applicable.

### BACKGROUND OF THE INVENTION

1. Field of Invention

This invention relates to an apparatus for releasably securing a thermistor. In this particular invention the securing apparatus includes a flexible foundation strap with an engaging strap extending therefrom. The apparatus also includes a securing strap secured to the foundation strap for releasably securing the distal end of the engaging strap to the foundation strap after the engaging strap has been received around at least a portion of a medical device.

2. Description of the Related Art

During the administering of medical care it is common for various medical devices to be secured to supporting structures near a patient, or secured to the patient's skin surfaces. For example, during procedures requiring intubation, it is desirable to secure the position of the tube(s) being used. Accordingly, it is a common practice to secure the tube(s) to the skin of the patient, proximate the point as which the tube(s) enter the body of the patient, with tape or other securing means. Further, it is common for intravenous (IV) medication bags to be supported in an elevated position near the patient, and for various monitoring devices to be supported near or on the patient for convenient access.

A thermistor is an electrical device that varies its resistance in relation to changes in temperature. With respect to infants, it is well known to secure a thermistor to the infant for monitoring the body temperature of the child. However, the thermistor must be periodically removed for transporting the infant for feeding, treating, testing, and other procedures. Under current practices, the thermistor is held in place under an adhesive patch. Repetitive removal of the patch from the infant causes irritation of the skin. Under some practices, a thermal gel is placed under the patch as well in order to achieve more accurate temperature readings. However, the thermal gel reduces the adhesion capabilities of the patch.

It is important that health care providers be able to perform medical procedures quickly and efficiently, and securing devices which are difficult and time consuming to use can unnecessarily complicate and/or delay the rendering of medical care. Certain previously known securing devices are disclosed in the following U.S. patents:

| Patent No. | Inventor(s) | Issue Date |
|---|---|---|
| Des. 310,721 | Biesang, III | Sept. 18, 1990 |
| 3,046,989 | Hill | July 31, 1962 |
| 3,826,254 | Mellor | July 30, 1974 |
| 3,927,676 | Schultz | Dec. 23, 1975 |
| 3,977,407 | Coleman et al. | Aug. 31, 1976 |
| 4,018,221 | Rennie | Apr. 19, 1977 |
| 4,088,136 | Hasslinger et al. | May 9, 1978 |
| 4,120,304 | Moor | Oct. 17, 1978 |
| 4,122,857 | Haerr | Oct. 31, 1978 |
| 4,142,527 | Garcia | Mar. 6, 1979 |
| 4,249,529 | Nestor et al. | Feb. 10, 1981 |
| 4,317,716 | Liller | Mar. 2, 1982 |
| 4,331,144 | Wapner | May 25, 1982 |
| 4,333,468 | Geist | June 8, 1982 |
| 4,351,331 | Gereg | Sept. 28, 1982 |
| 4,367,735 | Dali | Jan. 11, 1983 |
| 4,489,723 | Simons et al. | Dec. 25, 1984 |
| 4,548,200 | Wapner | Oct. 22, 1985 |
| 4,569,348 | Hasslinger | Feb. 11, 1986 |
| 4,583,976 | Ferguson | Apr. 22, 1986 |
| 4,617,017 | Hubbard et al. | Oct. 14, 1986 |
| 4,671,787 | Widman | June 9, 1987 |
| 4,690,675 | Katz | Sept. 1, 1987 |
| 4,702,736 | Kalt et at. | Oct. 27, 1987 |
| 4,744,358 | McGinnis | May 17, 1988 |
| 4,774,944 | Mischinski | Oct. 4, 1988 |
| 4,799,923 | Campbell | Jan. 24, 1989 |
| 4,822,342 | Brawner | Apr. 18, 1989 |
| 4,823,789 | Beisang, III | Apr. 25, 1989 |
| 4,836,200 | Clark | June 6, 1989 |
| 4,932,943 | Nowak | June 12, 1990 |
| 4,962,757 | Stefan | Oct. 16, 1990 |
| 4,976,700 | Tollini | Dec. 11, 1990 |
| 5,009,227 | Nieuwstad | Apr. 23, 1991 |
| 5,037,397 | Kalt et al. | Aug. 6, 1991 |
| 5,038,778 | Lott | Aug. 13, 1991 |
| 5,042,477 | Lewis | Aug. 27, 1991 |
| 5,135,506 | Genteliea et al. | Aug. 4, 1992 |
| 5,147,322 | Bowen et al. | Sept. 15, 1992 |
| 5,163,914 | Abel | Nov. 17, 1992 |
| 5,215,532 | Atkinson | June 1, 1993 |
| 5,306,233 | Glover | Apr. 26, 1994 |
| 5,368,024 | Jones | Nov. 29, 1994 |
| 5,755,698 | Kagan et al. | May 26, 1998 |

### BRIEF SUMMARY OF THE INVENTION

In one embodiment, the present invention provides a medical device securing apparatus for releasably securing at least one medical device to a supporting object or surface. The securing apparatus includes a foundation strap having first and second end portions and upper and lower surfaces. A securing mechanism for releasably securing said foundation strap to a supporting object is provided. In one embodiment, this securing mechanism is an adhesive surface portion provided on the lower surface of the foundation strap. An engaging strap extends outwardly from the second end portion of the foundation strap for being received about at least a portion of at least one medical device. The securing apparatus also includes a securing strap, secured at its proximal end portion to the foundation strap. The securing strap releasably engages at least a portion of the engaging strap, after the engaging strap has been received about at least a portion of at least one medical device. An adhesive securing mechanism is also provided for releasably securing the distal end portion of the engaging strap between the securing strap and the foundation strap, whereby the engaging strap is releasably held in position about at least a portion of at least one medical device.

In a preferred embodiment, the present invention is a holder for a thermistor especially for use with infants; however, the present invention is suitable for use with patients of all ages. The thermistor holder includes a foundation strap having first and second end portions and upper and lower surfaces. A securing mechanism for releasably securing said foundation strap to a supporting object is provided. In one embodiment, this securing mechanism is an adhesive surface portion provided on the lower surface of the foundation strap. A bridging member is carried by the foundation strap second end portion and carries an engaging strap for being releasably secured to the patient. The thermistor holder also includes a securing strap, secured at its proximal end portion to the foundation strap and releasably securable at its distal end to a portion of the engaging strap. The securing strap releasably engages at least a portion of the engaging strap, after the thermistor has been received between the securing strap and the bridging member. An insulative member is provided between the securing strap and the bridging portion to insulate the thermistor from ambient temperatures. The upper surface of the insulative member is provided with a reflective surface to further assist in the accuracy of the temperature readings acquired by the thermistor.

### BRIEF DESCRIPTION OF THE SEVERAL VIEWS OF THE DRAWINGS

The above mentioned features of the invention will become more clearly understood from the following detailed description of the invention read together with the drawings in which:

FIG. 1 illustrates a perspective view of a medical device securing apparatus of the present invention;

FIG. 2 illustrates a perspective view of a medical device securing apparatus of the present invention;

FIG. 3 illustrates a top plan view of a medical device securing apparatus of the present invention;

FIG. 4 illustrates a top plan view of a medical device securing apparatus of the present invention;

FIG. 5 illustrates a bottom view of a medical device securing apparatus of the present invention;

FIG. 6 illustrates a perspective view of a thermistor holder of the present invention showing the securing strap pulled away from the engaging strap for placement and/or removal of a thermistor;

FIG. 7 illustrates a perspective view of the thermistor holder of FIG. 6 showing the securing strap releasably secured to the engaging strap to secure the position of the thermistor; and

FIG. 8 illustrates an exploded bottom perspective view of the thermistor holder of FIG. 6.

### DETAILED DESCRIPTION OF THE INVENTION

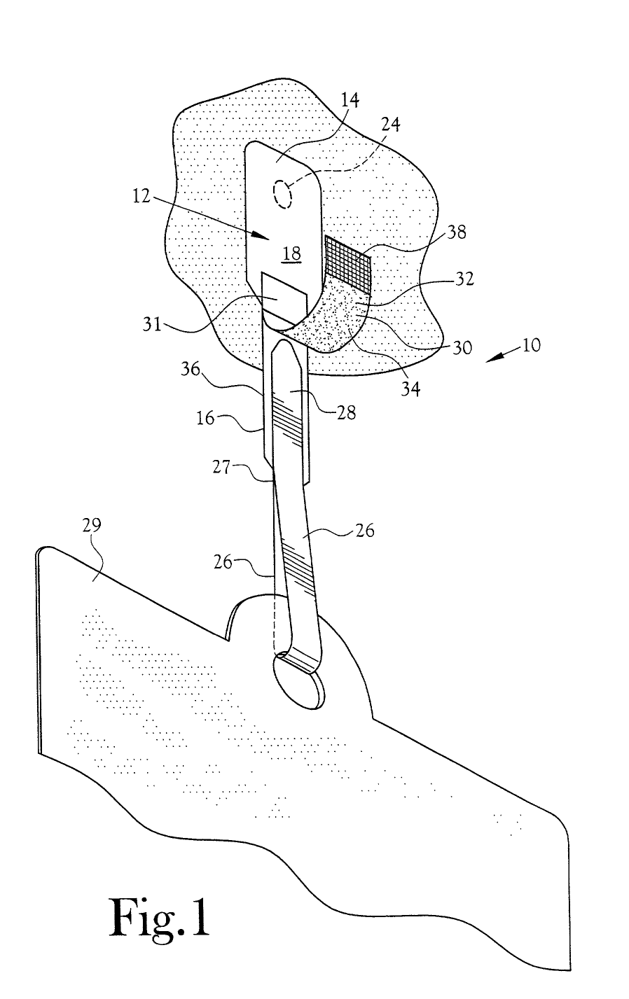

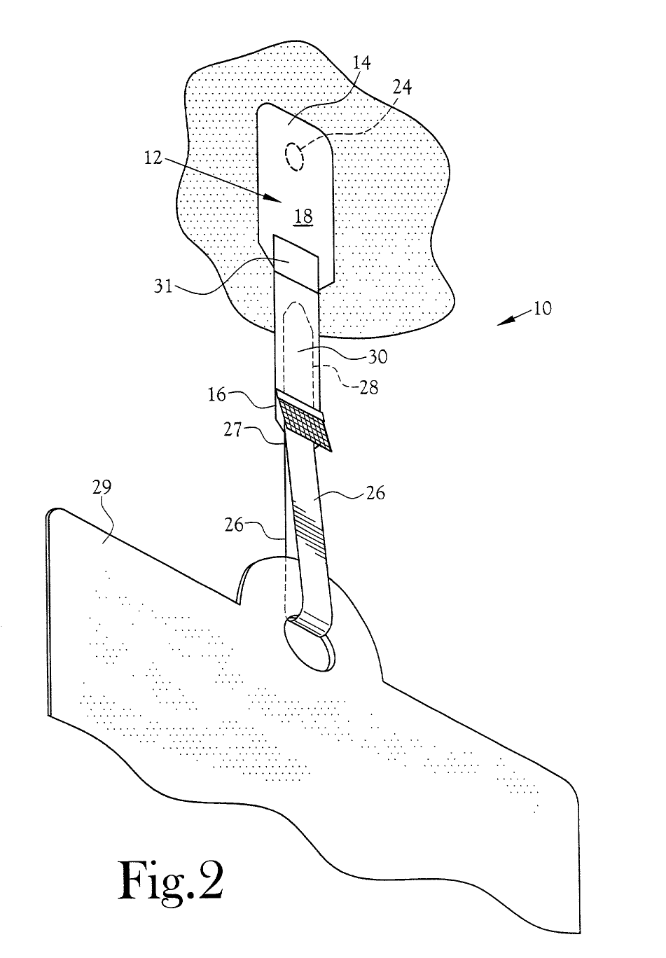

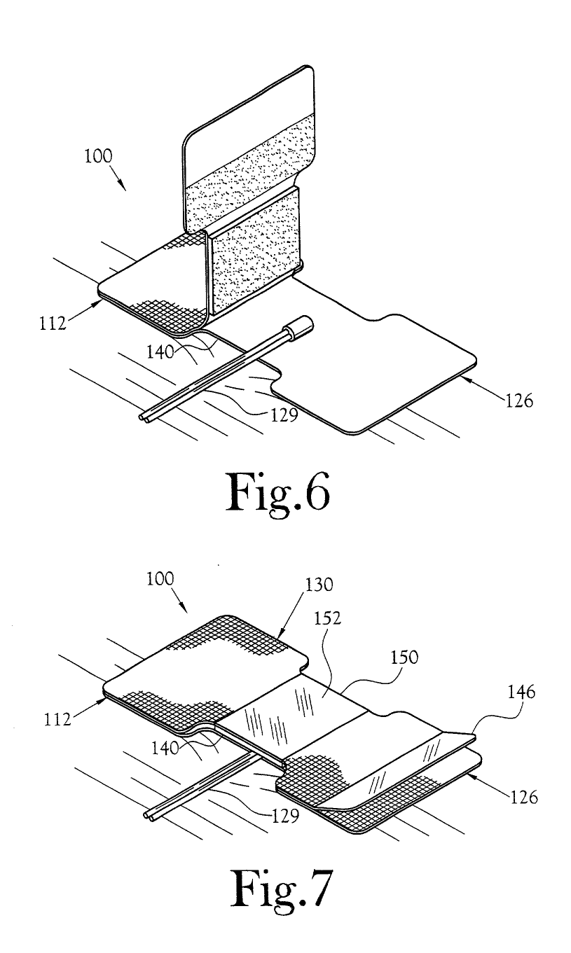

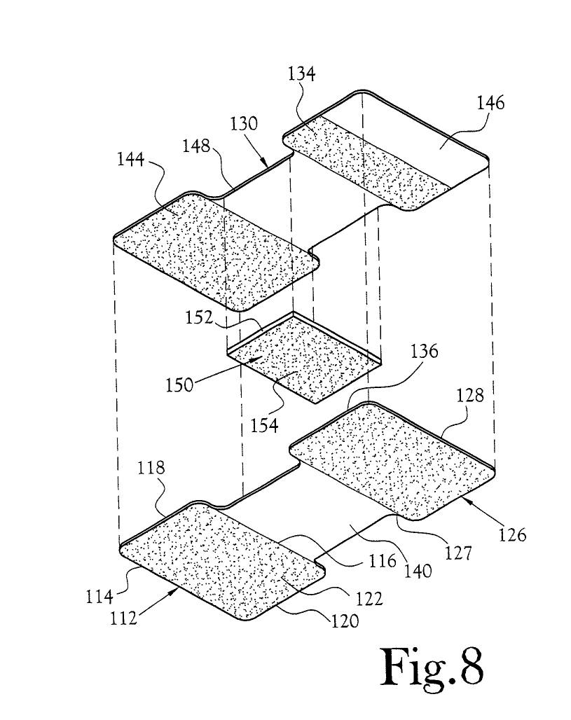

A medical device securing apparatus incorporating various features of the present invention is illustrated generally at 10 in the Figures. Whereas in FIGS. 1-2 the apparatus 10 is illustrated as being used to secure an IV medication bag 29 to a supporting surface, and whereas in FIGS. 6-8, the apparatus 100 is illustrated as securing the position of a thermistor 129, it is contemplated that the apparatus of the present invention can be used to secure various medical devices to other objects or supporting surfaces.

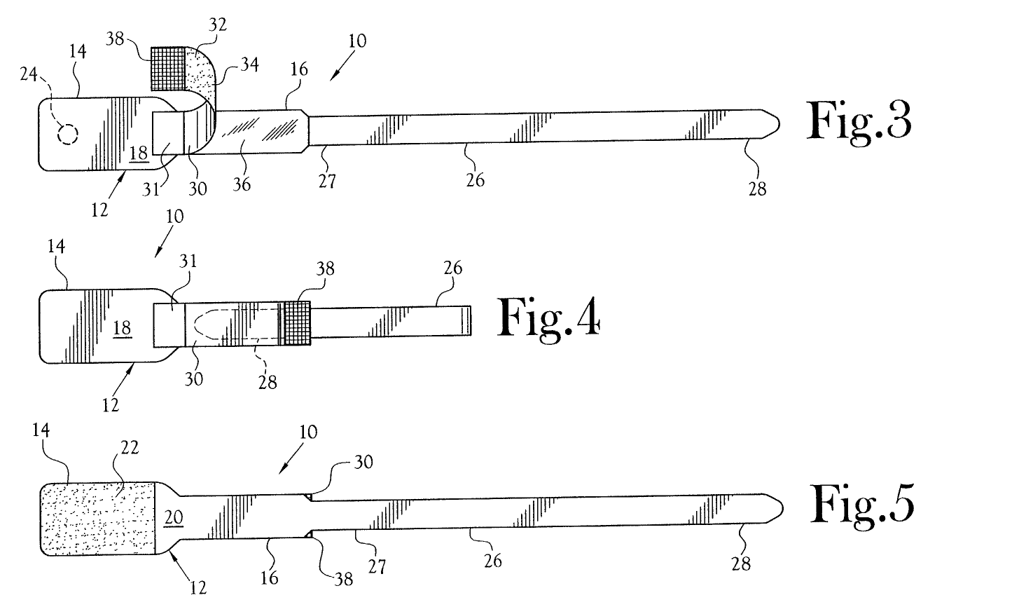

The medical device securing apparatus 10 includes a foundation strap 12. The foundation strap is preferably fabricated of a relatively thin, flexible material, such as, for example, 1.5 mil polyester or polyethylene. It will, however, be understood that other strong, durable fabricating materials can be used if desired. The foundation strap 12 has a first end portion 14 and a second end portion 16, and upper and lower surfaces 18 and 20, respectively. The first end portion 14 of the foundation strap 12 is provided with a securing mechanism for securing the foundation strap 12 to a supporting object. In the preferred illustrated embodiment of FIGS. 1-5 this securing mechanism includes an adhesive covered surface portion 22 disposed on the lower surface 20 of the foundation strap 12 proximate the first end portion 14 of the foundation strap 12. See FIG. 5. Utilizing the adhesive covered surface portion 22, the foundation strap 12 can be releasably bonded to various supporting objects and surfaces, including, without limitation, the skin of a patient, a wall surface, medical equipment, a patient bed, etc.

It is also contemplated that other securing mechanisms can be used, either alone or in combination with the adhesive covered surface portion 22, to secure the foundation strap 12 to a supporting object or surface. For example, the first end portion 14 of the foundation strap 12 can be provided with a hole for receiving a supporting hook or other supporting structure, such as the hole illustrated in phantom lines at 24 in FIGS. 1-3. It is also contemplated that various clamps and/or mechanical fasteners can be used as a securing mechanism.

The second end portion 16 of the foundation strap 12 carries a flexible engaging strap 26. The engaging strap 26 defines a proximal end portion 27 secured to the second end portion 16 of the foundation strap 12, and defines a distal end portion 28. Whereas the engaging strap 26 can be a separate component secured at its proximal end portion 27 to the foundation strap 12, it is contemplated that the engaging strap 26 can be integrally formed with the foundation strap 12 to simplify manufacture. The foundation strap 12 is also provided with a securing strap 30 for releasably engaging the engaging strap 26 after the engaging strap 26 has engaged the medical device(s) to be secured. In this regard, the engaging strap 26 is designed to be received through an opening in a medical device, or to be received around a medical device or a portion thereof.

Once the engaging strap 26 has been received about at least one medical device, or a portion thereof, the securing strap 30 is used to releasably secure the distal end portion 28 of the engaging strap 26 to the foundation strap 12 such that the engaging strap 26 is held in position about the medical device(s). More specifically, the securing strap 30 includes a proximal end portion 31 which is secured to the foundation strap 12, and defines an inner surface 32, for engaging the engaging strap 26. An adhesive bonding mechanism is also provided for releasably securing the distal end portion 28 of the tube engaging strap 26 to the securing strap 30. In the illustrated embodiment of FIGS. 1-5, the bonding mechanism includes an adhesive covered surface portion 34 provided on the inner surface 32 of the securing strap 30, and the securing strap 30 is disposed such that it releasably over lays a bonding surface 36 defined by the upper surface 18 of the foundation strap 12. The distal end portion 28 of the engaging strap 26 is secured in place by placing the distal end portion 28 on the bonding surface 36, and placing the securing strap 30 over the distal end portion 28 such that the adhesive covered surface portion 34 bonds to the distal end portion 28. In this embodiment the bonding surface 36 is preferably a smooth, non-porous surface which is wider than the distal end portion 28. Also, the securing strap 30 is preferably wider than the distal end portion 28. Accordingly, when the securing strap 30 is secured over the distal end portion 28, portions of the adhesive cover surface portion 34 engage the bonding surface 36 of the foundation strap 12 adjacent to the engaging strap 26, thereby firmly, yet releasably, locking the distal end portion 28 of the engaging strap 26 between the foundation strap 12 and the securing strap 30.

Notwithstanding the firm bonding of the engaging strap 26 between the foundation strap 12 and securing strap 30, the engaging strap 26 can be quickly and easily released by pulling back the securing strap 30 and disengaging it from the engaging strap 26 and the bonding surface 36. In this regard, in the preferred embodiment the securing strap 30 is provided with a pull tab member 38 at its distal end which is free of adhesive and which facilitates the grasping of the securing strap 30 after it has been adhesively bonded to the engaging strap 26 and the bonding surface 36.

FIGS. 6-8 illustrate a preferred embodiment of the present invention in which a thermistor 129 is releasably mounted to a patient, and especially an infant. The apparatus, or thermistor holder 100, includes a foundation strap 112. The foundation strap 112 has a first end portion 114 and a second end portion 116, and upper and lower surfaces 118 and 120, respectively. The first end portion 114 of the foundation strap 112 is provided with a securing mechanism for securing the foundation strap 112 to a supporting object. In the preferred illustrated embodiment of FIGS. 6-8 this securing mechanism includes an adhesive covered surface portion 122 disposed on the lower surface 120 of the foundation strap 112 proximate the first end portion 114 of the foundation strap 112.

The second end portion 116 of the foundation strap 112 carries a bridging member 140, which in turn carries a flexible engaging strap 126. The bridging member 140 defines a reduced width as compared to the foundation strap 112 and the engaging strap 126. The reduced width of the bridging portion 140 assists in drawing the portions of the patient's skin together in order to effectuate an accurate temperature reading via the thermistor 129. The engaging strap 126 defines a proximal end portion 127 secured to the bridging member 140, and defines a distal end portion 128. Whereas the engaging strap 126 can be a separate component secured to the bridging member 140, and the bridging member 140 can be a separate component secured to the foundation strap 112, it is contemplated that the engaging strap 126, the bridging member 140 and the foundation strap 112 are integrally formed as illustrated to simplify manufacture.

A securing strap 130 is provided for releasably engaging the engaging strap 126 after the engaging strap 126 has been engaged on the patient's skin and the thermistor 129 has been inserted between the securing strap 130 and the engaging strap 126. The securing strap 130 defines a configuration similar to that of the foundation strap 112, bridging member 140 and engaging strap 126. The proximal end 142 of the securing strap 130 defines an adhesive covered surface portion 144 corresponding to the upper surface 118 of the foundation strap 112. A second adhesive covered surface portion 134 is defined by the distal end 132 of the securing strap 130 for releasably securing the distal end 132 to the proximal end portion 127 of the engaging strap 126. A free end 146 is defined at the distal end 132 of the securing strap 130 to define a means for grasping the securing strap 130 in order to release the securing strap 130 from the engaging strap 126. In one embodiment, the adhesive covered surface of the securing strap 130 extends continuously from the adhesive covered surface portion 144 to the second adhesive covered surface portion 134, forming a contiguous adhesive covering on the securing strap 130.

An insulative member 150 is carried on the lower surface of a central portion 148 of the securing strap 130. One material found effective for fabricating the insulative member 150 is an insulative foam rubber. However, it will be understood that other materials may be used as well. The insulative member 150 is provided for insulating the thermistor 129 from ambient temperatures, in order to obtain a more accurate temperature reading from the patient. In order to further assist in obtaining an accurate temperature reading, the upper surface 152 of the insulative member 150 defines a reflective surface. One such reflective surface 152 is accomplished by means of a reflective metallic material such as foil disposed between the insulative member 150 and the securing strap 130. The lower surface 154 of the insulative member defines an adhesive coated surface for securing the thermistor 129 thereto, as well as for releasably securing the insulative member 150 to the upper surface of the bridging member 140.

Notwithstanding the firm bonding of the securing strap 130 to each of the foundation strap 112 and the engaging strap 126, the securing strap 130 can be quickly and easily released from the engaging strap 126 by pulling back the free end 146 defined by the distal end 132 of the securing strap 130 and disengaging it from the engaging strap 126.

In light of the above it will be recognized that the present invention provides a medical device securing apparatus having great advantages over the prior art. The securing apparatus 10, 100 of the present invention can be quickly and easily secured to various supporting object or surfaces, and quickly and easily secured to various medical devices. The medical device which is secured can also be quickly and easily released by simply disengaging the securing strap 130.

While a preferred embodiment has been shown and described, it will be understood that it is not intended to limit the disclosure, but rather it is intended to cover all modifications and alternate methods falling within the spirit and the scope of the invention as defined in the appended claims.

Having thus described the aforementioned invention, I claim:

1. A thermistor holder for releasably securing a thermistor to a patient, said thermistor holder comprising:
a foundation strap having first and second end portions and upper and lower surfaces;
a first securing mechanism carried by said foundation strap for releasably securing said foundation strap to a patient;
a bridging member carried by and extending outwardly from said foundation strap second end;
an engaging strap carried by and extending outwardly from said bridging member for releasably engaging the patient, said engaging strap defining a first end and a second end, said engaging strap first end being secured to said bridging member;
a second securing mechanism carried by said engaging strap for releasably securing said engaging strap to a patient;
a securing strap for releasably engaging the thermistor and for engaging at least a portion of said engaging strap when at least a portion of the thermistor is disposed between said bridging member and said securing strap, said securing strap defining a first end and a second end, said securing strap first end being secured to said foundation strap and said securing strap second end defining a free end relative to said engaging strap;
a third securing mechanism carried by at least one of said foundation strap and said securing strap first end for securing said securing strap in engagement with said foundation strap; and
a forth securing mechanism carried by at least one of a portion of said securing strap second end and said engaging strap for releasably securing said securing strap second end to said engaging strap in order to secure at least a portion of the thermistor between said bridging member and said securing strap.

2. The thermistor holder of claim 1 further comprising an insulative member disposed between said securing strap and said bridging member for insulating the thermistor from ambient temperatures.

3. The thermistor holder of claim 2 wherein said insulative member defines a reflective upper surface for reflecting heat away from the thermistor.

4. The thermistor holder of claim 1 wherein each of said foundation strap and said engaging strap defines a first width, and wherein said bridging member defines a second width, said second width being more narrow than said first width.

5. A thermistor holder for releasably securing a thermistor to a patient, said thermistor holder comprising:
a foundation strap having first and second end portions and upper and lower surfaces, said foundation strap defining a first width;
a first securing mechanism carried by said foundation strap for releasably securing said foundation strap to a patient;
a bridging member carried by and extending outwardly from said foundation strap second end, said bridging member defining a second width smaller than said first width;
an engaging strap carried by and extending outwardly from said bridging member for releasably engaging the patient, said engaging strap defining a first end and a second end, said engaging strap first end being secured to said bridging member, said engaging strap defining said first width;
a second securing mechanism carried by said engaging strap for releasably securing said engaging strap to a patient;
a securing strap for releasably engaging the thermistor and for engaging at least a portion of said engaging strap when at least a portion of the thermistor is disposed between said bridging member and said securing strap, said securing strap defining a first end and a second end, said securing strap first end being secured to said foundation strap and said securing strap second end defining a free end relative to said engaging strap;
a third securing mechanism carried by at least one of said foundation strap and said securing strap first end for securing said securing strap in engagement with said foundation strap;
a forth securing mechanism carried by at least one of a portion of said securing strap second end and said engaging strap for releasably securing said securing strap second end to said engaging strap in order to secure at least a portion of the thermistor between said bridging member and said securing strap; and
an insulative member disposed between said securing strap and said bridging member for insulating the thermistor from ambient temperatures.

6. A thermistor holder for releasably securing a thermistor to a patient, said thermistor holder comprising:
a foundation strap having first and second end portions and upper and lower surfaces, said foundation strap defining a first width;
a first securing mechanism carried by said foundation strap for releasably securing said foundation strap to a patient;
a bridging member carried by and extending outwardly from said foundation strap second end, said bridging member defining a second width smaller than said first width;
an engaging strap carried by and extending outwardly from said bridging member for releasably engaging the patient, said engaging strap defining a first end and a second end, said engaging strap first end being secured to said bridging member, said engaging strap defining said first width;
a second securing mechanism carried by said engaging strap for releasably securing said engaging strap to a patient;
a securing strap for releasably engaging the thermistor and for engaging at least a portion of said engaging strap when at least a portion of the thermistor is disposed between said bridging member and said securing strap, said securing strap defining a first end and a second end, said securing strap first end being secured to said foundation strap and said securing strap second end defining a free end relative to said engaging strap;
a third securing mechanism carried by at least one of said foundation strap and said securing strap first end for securing said securing strap in engagement with said foundation strap;
a forth securing mechanism carried by at least one of a portion of said securing strap second end and said engaging strap for releasably securing said securing strap second end to said engaging strap in order to secure at least a portion of the thermistor between said bridging member and said securing strap; and
an insulative member disposed between said securing strap and said bridging member for insulating the thermistor from ambient temperatures, said insulative member defining a reflective upper surface for reflecting heat away from the thermistor.
